(19) United States
(12) Patent Application Publication
(10) Pub. No.: US 2002/0119191 A1
(43) Pub. Date: Aug. 29, 2002

(54) Pharmaceutical or food composition for treatment or prevention of brain edema

(76) Inventors: Hitoo Nishino, Nagoya-shi (JP); Kunio Torii, Kawasaki-shi (JP); Hisayuki Uneyama, Kawasaki-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 09/556,701

(22) Filed: Apr. 24, 2000

(51) Int. Cl. A61K 9/54; A61K 9/00

(52) U.S. Cl. 424/458; 424/400

(57) ABSTRACT

Melatonin has an activity of treating or preventing brain edema. Thus, the invention relates to a pharmaceutical composition comprising melatonin as an active ingredient. And, the invention relates to use of melatonin in the preparation of a pharmaceutical composition comprising melatonin as an active ingredient.

### FIELD OF THE INVENTION

The present invention relates to compositions for the treatment or prevention of brain edema. More particularly, it relates to pharmaceutical or food compositions for the treatment or prevention of brain edema comprising melatonin as an active ingredient. And, it relates to the use of melatonin in the preparation of the above composition. It relates also to a method for the treatment or prevention of brain edema with the above composition.

### BACKGROUND OF THE INVENTION

Brain edema refers to a condition where fluid is excessively accumulated in brain parenchyma (in intercellular spaces or in cells) resulting in swell of brain tissue. The swell of tissue in the limited cranial space increases intracranial pressure. Thus, the brain edema generally associates with increased intracranial pressure.

Brain edema can be etiologically classified into “vasogenic brain edema” and “cytotoxic brain edema” (I. Klatzo, J. Neuropatho. Exp. Neurol., 25: 1-14, 1967).

Vasogenic edema is caused by an injury of a cerebral blood vessel. The injury of cerebral capillaries modifies and deteriorates the vascular permeability. When the vasopermeability is modified, fluid migrates into intracellular spaces of brain resulting in the increase of a fluid content in the intracellular spaces. The vasogenic edema is often found in brain tumor, cerebral hemorrhage and the like.

Cytotoxic edema is caused by an injury of cells. The injury of cells modifies and deteriorates the permeability of cell membrane. When the cell membrane permeability is modified, fluid migrates into cells resulting in the increase of a fluid content in the cells. The cytotoxic edema is often found in hypoxia, toxipathy (induced by arsenium, carbon oxide and the like) and metabolic disorders (diabetic coma, uremia and the like).

Apart from the above two types of edema, brain edema caused by brain ischemia or deficient cerebral blood flow is called as “ischemic brain edema”. Brain edema caused when a cerebral blood is recirculated after brain ischemia or cerebral blood flow deficiency is called as “post-ischemic brain edema”. The modification of vascular or cellular permeability would also be involved in the onset of the edema of this classification.

Once a subject was suffered from brain edema irrespective of the cause of deterioration of vascular permeability or the cause of deterioration of cellular permeability, the brain edema itself leads to a secondary disorder such as a disturbance of a cerebral blood flow, ischemia, hypoxia, cerebral hernia and the like due to the increase in intracranial pressure. And, the secondary disorder gives an additional deterioration of vascular or cellular permeability and as the results, extends the edema. Such an extension of edema by edema itself looks like a forest fire where a fire spread itself from one wood to other wood (see I. Klatzo & F. Seitelberger (eds): Brain Edema, Springer-Verlag, New York, 1967). This is inherent and characteristic in brain edema caused by the presence of brain tissue in a rigid and limited cranial space which differs from other organs. Due to such a “forest fire-like” extension, brain edema is a severe mortal disease.

The treatment of brain edema relies on the administration of a hypersonic solution, a steroid, a diuretic and an adjuvant such as a thrombolytic and a microcirculation improver. The hypertonic solution mainly comprises glycerol or mannitol (for example, 10% of glycerol, 5% of fructose and 0.9% of NaCl) and acts to migrate a fluid in a brain tissue into a blood vessel by increasing a serum osmotic pressure. The steroid is considered to exhibit an anti-brain edema effect by reinforcing a cell membrane and the like.

With the increase in the population of the aged, the population of subjects suffering from diseases in which brain tissues are injured such as cerebral thrombosis, cerebral embolism and cerebral infarction and subjects suffering from brain edema will be increased. Consequently, the treatment and prevention of brain edema is a problem to be urgently solved.

Melatonin (N-[2-(5-methoxy-1H-indol-3-yl)ethyl]acetamide) is secreted from pineal gland which is one of neurohormonal organs and it influences the formation of a diurnal rhythm (for example, Chem. & Eng. News, 45: 40, 1967).

Since melatonin has the above physiological action, it is used for the treatment of the disorder of a diurnal rhythm showing various disorders such as a sleep disorder, an emotional disorder, an immune hypofunction and the like caused by a muzziness by the difference in time and other causes (for example, Barchas et al., Nature 214: 919, 1967 and A. Miles et al., CRC Crit. Rev. Clin. Lab. Sci., 25: 231-253, 1987).

Melatonin has an anti-oxidative activity. For example, it prevents in vitro an oxidative deterioration by oxygen free radicals in various biocomponents (R. J. Reiter et al., Life Sci., 60(25), 2255-2271, 1997).

R. J. Reiter describes that oxygen free radicals may be involved in a deterioration of nervous system of the aged and such a deterioration may be reduced by an anti-oxidative activity of melatonin (R. J. Reiter, FASEB J., 9: 526-533, 1995).

Further, it is pointed out that melatonin administered inhibits the production of NO in brain after transient ischemia/recirculation and reduces a brain injury caused by free radicals (J. M. Guerrero et al., J. Pineal Res., 23: 24-31, 1997).

Sunghee Cho et al., Brain Res., 755(2), 335-338, 1978 describes that melatonin intraperitoneally administered, especially prior to cerebral ischemia or during recanalization, protects CA1 hippocampal neurons from an ischemic injury.

In addition, it has been reported that in models of brain ischemia induced by ligating a middle cerebral artery, brain necroses (by observation of tissues under a microscope) in rats having no detectable level of melatonin after pinealectomy are significantly larger than in normal rats (Hari Manev et al., FASEB J, 10(13), 1546-1551, 1996).

WO 97/20555 discloses that a mild motor dysfunction such that a foot-fault rate is 0.01 due to brain ischemia can be lowered to the foot-fault rate of 0.002 by administration of a “rescue solution” containing melatonin, kynurenine and others to a brain ischemic rat. The foot-fault rate of the group without the administration of the rescue solution was 0.05. (The foot fault rate is determined according to Hernandez-Schallert foot-fault test wherein rats forcedly walk on a bar having 3 to 6 cm in diameter and the number of missed (slipped) rats is counted. The foot-fault rate is a proportion of missed rats after ischemia to missed rats before ischemia.)

On the other hand, it has been known that orally administered melatonin migrates in blood (A. L. Elizabeth et al., J. Clin. Endocrinol. Metab., 61: 1214-1216, 1985; O. Vakkuri et al., Life Sci., 37: 489-495, 1985; M. Aldhous et al., Br. J. Clin. Pharmacol., 19: 517-521, 1985; and F. Waldhauser et al., Neuroendocrinology, 39: 307-313, 1984).

And, it has been known that intravenousy administered melatonin migrates in brain (P. A. Vitte et al., Pineal Res. 5: 437-453, 1988 and D. L. Baris et al., Int. J. Rds. Appl. Instrum. [B] 18: 357-362, 1991).

As to edema, S. Bertuglia et al., Cardiovascular Research, 31: 947-952, 1996 describes that the administration of melatonin reduces edema caused by ischemia-reperfusion of a microcirculation in a cheek pouch of a hamster. According to this article, melatonin reduces the increase of permeability of capillaries caused by free radicals (produced by topically exposing to hypoxanthine-xanthine oxidase). And, S. Cruzzocrea et al., J. Pineal Res., 23: 106-116, 1997 describes that the administration of melatonin suppresses a carrageenan-induced inflammatory paw swelling in rats. It is suggested that a control of an expression of an inducible NO synthase and a scavenging action of a free radical peroxynitrite are involved in the above inhibition. Y. Oyanagui, Inflammation, 21: 643-654, 1997 describes that various antioxidants including melatonin enhance or prolong suppression by dexamethasone of an ischemic or histamine-induced paw edema. Further, W. Qi, et al, Dig. Dis. Sci., 44: 2257-2262, 1999 describes that melatonin reduces an acute pancreatitis (pancreatic edema) induced by cerulein in rats. It is supposed that a radical scavenging action of melatonin is involved in the above reduction of edema.

The above local inflammatory edema would be caused also by the modification in vasopermeability (caused by inflammatory or the like) or the modification in cell membrane permeability (caused by free radicals or the like).

However, a cerebral vasopermeability is controlled by a blood-brain barrier which is absent in blood vessels in other organs. Particularly, astroglia is involved in the blood-brain barrier, which is also absent in blood vessels in other organs. Studies of brain edema in view of the brain specific vasopermeability has not been satisfactorily conducted.

Further, a study from the view point of a “forest fire-like” extension of brain edema which is caused by the presence of brain tissue in the limited space via the brain-specific blood-brain barrier and astroglia has not been fully studied.

In order to treat and prevent brain edema more effectively, a further study from a new and different viewpoint will be necessary.

### SUMMARY OF THE INVENTION

Accordingly, an object of the invention is to provide an useful means for the treatment or prevention of brain edema more effectively.

One aspect of the invention relates to a pharmaceutical or food composition for the treatment or prevention of brain edema comprising melatonin in an effective amount for said treatment or prevention.

Another aspect of the invention is the use of melatonin in the preparation of a pharmaceutical or food composition for the treatment or prevention of brain edema comprising melatonin in an effective amount for said treatment or prevention.

A further aspect of the invention relates to a method for the treatment or prevention of brain edema comprising administering to a subject in need of said treatment or prevention, a pharmaceutical or food composition for said treatment or prevention comprising melatonin in an effective for said treatment or prevention.

### BRIEF EXPLANATION OF DRAWINGS

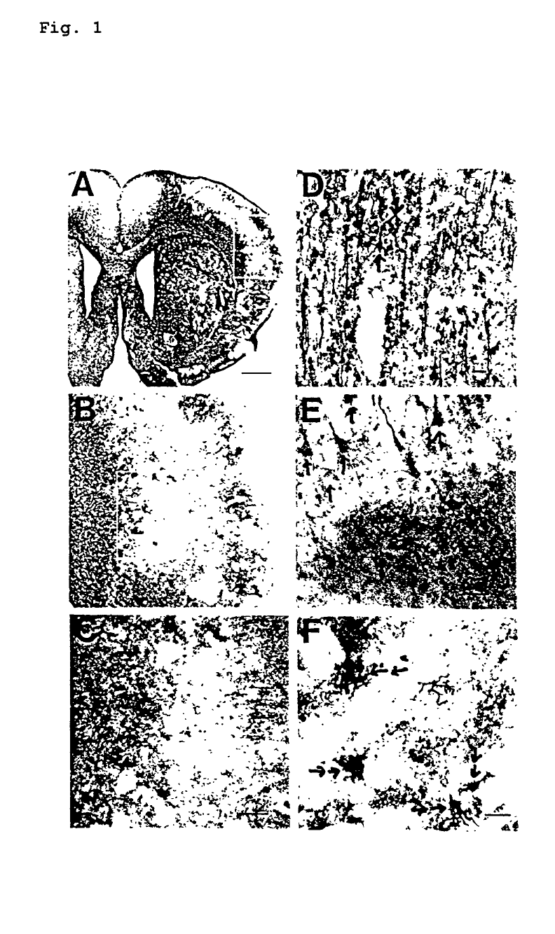

In FIG. 1, photograph A is a micrograph of a specimen of brain after brain ischemia, said specimen being stained with GFAP. Photograph B is an amplification of an area enclosed with a square in photograph A, photograph C is an amplification of an area enclosed with a square in photograph B, and photograph D is an amplification of an area enclosed with a square in photograph A. Scale as indicated at the lower right in photograph A is 1 mm and that in each of photographs B to E is 50 μm. In photographs, → represents a neuron and →→ represents a neuron and an astroglia.

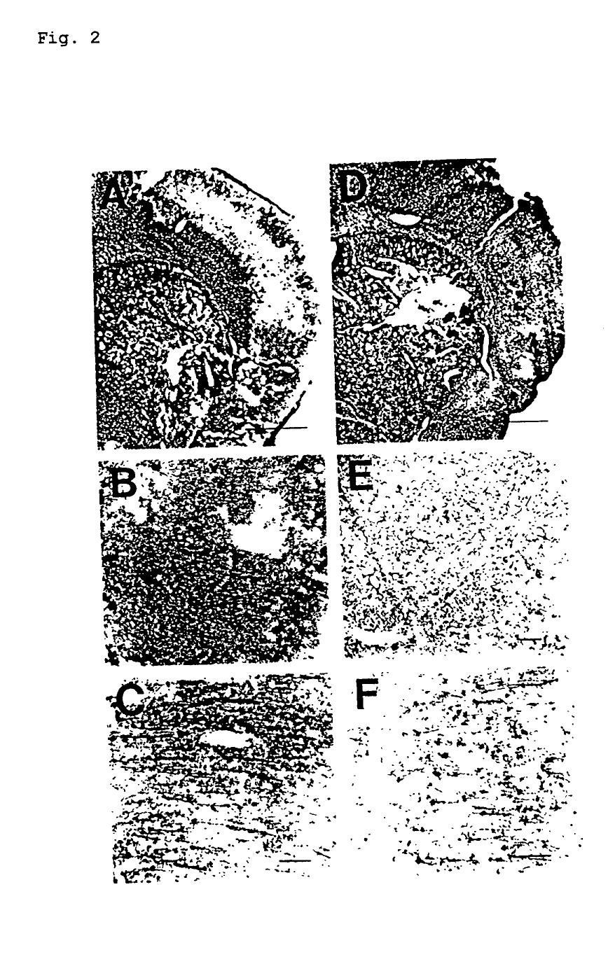

In FIG. 2, photographs A and B each is a micrograph of a specimen of brain after brain ischemia in the non-melatonin administration group, said specimen being stained with DFAP. Photographs D and E each is a micrograph of a specimen of brain after brain ischemia in the melatonin administration groups, said specimen being stained with DFAP. Photograph C is a micrograph of a specimen of brain after brain ischemia in the non-melatonin administration group, said specimen being stained with MAP-2. Photograph F is a micrograph of a specimen of brain after brain ischemia in the melatonin administration groups, said specimen being stained with MAP-2.

### DETAILED EXPLANATION OF THE INVENTION

During our studies concerning the treatment and prevention of brain edema, our attention was directed to a blood-brain barrier deeply involved in a “forest fire-like” extension of brain edema and inherent in a cerebral blood vessel, especially astroglia characteristic in a blood-brain barrier. Then, our studies was directed to the blood-brain barrier, especially an injury of astroglia, its protection and a treatment of injured astroglia.

As the results of the above studies, we obtained the following two findings, based on which the invention has been completed.

The first finding is that brain ischemia preferentially injures astroglia to neuron (FIG. 1). The injury of astroglia will be directly associated with an injury of a blood-brain barrier, that is, a modification in a permeability of a cerebral blood vessel. Then, the first injury of astroglia will cause brain edema, and the brain edema will secondary generate deficiency of cerebral blood flow. The second deficiency of cerebral blood flow will secondary injure other healthy astrocytes expanding the brain edema.

The second finding is that melatonin preferentially protects astroglia from an injury caused by a poison or ischemia and preferentially cures an injured astroglia (FIG. 2). The protection and curing of astroglia by melatonin will directly prevent the vascular permeability from injury and cures the injury.

As the results of further studies based on the above two findings, we found that melatonin treats and prevents brain edema, or inhibit a “forest fire-like” extension of brain edema through its activity capable of protecting astroglia from injury and curing of injured astroglia.

The preferential activity of melatonin for astroglia cannot be explained from an anti-oxidative activity of melatonin suggested to be involved in a local inflammatory edema as described in many literatures.

The first group of subjects received with the pharmaceutical or food composition of the invention comprising melatonin are patients suffering from brain edema. According to our study, in the first group of the subjects, melatonin treats astroglia which has been injured by ischemia, edema or other causes and treats a blood-brain barrier so that edema is cured. And, melatonin prevents or reduces a secondary injury of a blood-brain barrier caused by a secondary injury of astroglia due to brain edema so that a “forest fire-like” extension of brain edema is suppressed. Accordingly, the composition of the invention is used for the treatment of brain edema in the first group of the subjects.

The second group of subjects received with the pharmaceutical or food composition of the invention are subjects who are judged by clinicians to have the risk of suffering from brain ischemia. According to our finding, such subjects have the risk of injuring their astroglia and blood-brain barrier. In the second group of the subjects, melatonin will protect the astroglia and blood-brain barrier from injury caused by brain ischemia so that an onset of brain edema is prevented. Accordingly, the composition of the invention is used for the prevention of brain edema in the second group of the subjects.

The subjects having the risk of suffering from brain ischemia include, but is not limited to, subjects suffering from cerebral thrombosis, cerebral embolism, cerebral infarction, cerebral hemorrhage, subarachnoid hemorrhage, transient brain ischemia, hyperlipemia, hypertension, cardiac arrest or brain contusion.

Diagnosis and monitoring of brain edema is conducted by a clinician based on one or more informations including a progress of conditions such as an increase in intracranical pressure, a CT scanning, a MRI and the like. That is, the brain edema of this invention depends on and defined as the result of a clinician's judgement based on the above diagnosis or monitoring.

Melatonin as used herein include that encapsulated in an encapsulating matrix, a liposome or the like.

Melatonin encapsulated in an encapsulating matrix is gradually released from the matrix into blood so that its residence time in blood is apparently increased.

The encapsulating matrix comprises cyclodextrin and pharmaceutically acceptable biodegradable synthetic polymers such as polylactic acid, a copolymer of lactic acid and glycol, poly-β-hydroxybutyric acid and the like. Cyclodextrins attached onto a pharmaceutically acceptable synthetic polymer are included in the definition of cyclodextrin.

When melatonin is encapsulated in a liposome, its residence time in blood can be also increased.

As methods for the preparation of liposomes, various methods such as a vortex method (Bangham AD et al., Methods Membr. Biol., 1: 1-20, 1974), an ultrasonic treatment method (Johnson S M et al., Biochim. Biophys. Acta, 233: 820-826, 1971), an ethanol injection method (Kremer J M H et al., Biochemistry, 16: 3932-3941, 1980), a french press method (Hamilton et al., J. Lipid Res. 21: 981-982, 1980), a cholic acid removal method (Enoch H G et al., Proc. Natl. Acad. Sci., 76: 145-149, 1979), an ether injection method (Deamer D N, Ann, N.Y. Acad. Sci., 308: 250-258, 1987), a freeze-thaw method (Papahadjopoulos D et al., Biophim. Biophys. Acta, 394: 483-491, 1975) and a reverse phase evaporation method (Szoka F et al., Proc. Natl. Acad. Sci. USA, 75: 4191-4198, 1978) and the like have been known. Their disclosures with respect to the methods for the preparation of liposomes are incorporated by reference herein.

The composition of the invention may be either a pharmaceutical composition or a food composition. The pharmaceutical and food compositions essentially contain melatonin in an amount effective for treatment or prevention of brain edema.

It is desirable to administer the composition of the invention to a subject as soon as possible when brain ischemia was found. If the administration is delayed, brain edema will be generated and extended. By the early administration, it is possible to prevent an onset of brain edema. The composition of the invention is administered to a subject having the risk of suffering from brain ischemia and then brain edema every day or at a predetermined interval depending on condition or state of a diseases such as cerebral thrombosis or cerebral embolism.

The administration of the composition of the invention is effective also for the treatment of a subject suffering from brain edema. The administration of the composition of the invention is continued until the risk of an onset of brain edema is lowered or brain edema is reduced to a desirable level. It is stopped by a clinician's judgement.

An amount of melatonin to be administered to the subject in need of the treatment or prevention of brain edema is varied with various factors including sex, age, body weight and diet of a subject to be administered; an administration route; condition of brain edema; degree of risk of inducing brain ischemia; condition of diseases such as cerebral thrombosis and cerebral embolism; condition of circulatory systems; and the like. It is determined by a clinician totally considering the above informations.

When the pharmaceutical composition of the invention is administered for the prevention of brain edema, its daily dose is determined such that a daily blood concentration of melatonin ranges from 1 ng/ml to 100 μg/ml. When the pharmaceutical composition of the invention is administered for the treatment of brain edema, its daily dose is determined such that a daily blood concentration of melatonin ranges from 10 ng/ml to 300 μg/ml.

Since an availability of melatonin is varied whether melatonin is administered by orally or i.v. injection or whether melatonin is encapsulated or not, especially with a dosage form for oral administration, a daily dose of the pharmaceutical composition is naturally varied.

The expression “daily blood concentration” as used above means a total blood concentration of melatonin administered per day. For example, when the blood concentration by the first administration reaches to 50 μg/ml and the blood concentration by the second administration on the same day reaches to 30 μg/ml, the daily blood concentration is calculated to be 80 μg/ml. It is unnecessary to determined the daily blood concentration by practically measuring the blood concentration of melatonin. It is estimated by a clinician or a specialist based on informations previously given, from which a daily dose can be determined by the clinician and the specialist.

Preferably, the oral dose is determined based on a migration ratio of melatonin orally administered to blood. To give 100 ng/ml of blood concentration of melatonin, an oral dose of about 0.8 mg/kg-body weight would be necessary.

The above-mentioned blood concentration of melatonin is referred to melatonin in a free form. In case melatonin is encapsulated in an encapsulating matrix or a liposome, its apparent residence time in blood is longer since melatonin in an encapsulated form is gradually released in blood. Thus, the blood concentration of melatonin in the encapsulated form may be lower than that in melatonin in the free form.

The pharmaceutical composition of the invention is administered by various routes, such as permucosally (sublingually, intranasally, oral mucosally and the like), orally, enterally, percutaneously, intravenously, by aspiration, by suppository or by instillation. An administration route is determined depending on an amount of melatonin to be administered, conditions of a patient or a subject and the like. It is determined by a clinician.

In addition to melatonin, the pharmaceutical composition of the invention contains a pharmaceutical carrier which is varied depending on its dosage form. The pharmaceutical carrier should be pharmaceutically acceptable and has no or little pharmaceutical activity in vivo.

When the pharmaceutical composition of the invention is orally administered, binders such as tragacanth gum, acacia, corn starch, gelatin and the like; vehicles such as potassium diphosphate and the like; disintegrators such as corn potato, potato starch, alginic acid and the like; lubricants such as magnesium stearate and the like; sweetening agents such as sucrose and the like; dyes; perfumes such as orange flavor and the like; solvents such as water, ethanol, glycerol and the like can be suitably used as the pharmaceutical carrier.

When the pharmaceutical composition of the invention to be orally administered contains a pharmaceutically acceptable antioxidant such as cysteine, glutathione, ascorbic acid, sodium metasulfite, sodium bisulfite or the like as the pharmaceutically acceptable carrier, favorable results may be obtained.

The pharmaceutical composition for injection of the present invention may be a sterile powder composition, a freeze dried powder composition or the like which can be used by merely dissolving in a sterile water.

A diluent or a solvent such as a sterile water, an isotonic saline, a pH butter and the like can be used as a pharmaceutically acceptable carrier used in the pharmaceutical composition for injection. An aqueous ethanol may be used as the solvent.

The pharmaceutical composition for injection of the invention may contain saccharides such as glucose, mannitol, dextran and the like; polyhydric alcohols such as glycerol and the like; inorganic salts such as sodium salt, magnesium salt and the like as a pharmaceutical carrier. Further, it may contain a pharmaceutically acceptable antioxidant such as cysteine, glutathione, ascorbic acid, sodium metasulfite, sodium bisulfite and the like.

Pharmaceutical carriers contained in dosage forms for other administration routes such as intranasal, aspiration or percutaneous administrations are known for those skilled in the art.

The dosage forms and the pharmaceutical carriers mentioned above are known in the art and described in, for example, Reimington's Pharmaceutical Science, ed. 16 (1980), Mack Publishing Company, which is incorporated herein by reference.

The pharmaceutical composition of the invention may contain any agents in addition to melatonin. For example, when the pharmaceutical composition of the invention is administered orally or parenterally, it may contain nutrients such as amino acids, vitamins, lipids, glucose and the like. When the pharmaceutical composition of the invention is administered by instillation, it may contain nutrients such as glucose, vitamins, amino acids, lipids and the like.

Further, the pharmaceutical composition of the invention may contain therapeutic agents conventionally used in the treatment of brain edema, that is, one or more therapeutic agents selected from hypertonic solutions such as glycerol, mannitol and the like; steroids such as dexamethasone and the like; diuretics such as furosemide, acetazol and the like; and adjuvants such as an thrombolytic agent, microcirculation improvers, and urinastin and gabexate mesilate stabilizing cell membranes.

Further, the pharmaceutical composition of the invention may contain therapeutic agents for the treatment of diseases such as cerebral thrombosis, cerebral embolism, a hypertensive encephalopathy. The known therapeutic agents for the treatment of cerebral embolism includes an anti-edema, an anticoagulant, a thrombolytic agent, a calcium antagonist and the like. And, the known therapeutic agents for the treatment of cerebral thrombosis includes an anti-edema, an anti-platelet agent, a calcium antagonist and the like.

The pharmaceutical composition of the invention for oral administration is preferably in the form of sustained release. For the sustained release, standard sustained released preparations such as a preparation having a gel coating and a preparation having a multiple coating and preparations for local release such as a preparation capable of rupturing in a pylorus or a preparation capable of foaming in a duodenum are well known. Examples of the composition for oral administration include tablets, pills, capsules, ampuls, folded powders, elixirs, suspensions, syrups and the like.

The pharmaceutical composition of the invention for per rectal administration may be in the form of a suppository. Various forms of suppositories are well known.

When the composition of the invention is a food composition, any food may be used since melatonin is tasteless and stable against heat and enzymes under cooking conditions. The food includes cooked foods such as a hamburger and a soup as well as uncooked foods such as a fruit juice. Cold foods such as an ice cream, emulsified foods such as a mayonnaise, gelled foods such as a custard pudding and a jelly and fermented food such as yogurt are included in the food of the invention.

Foodstuffs, for example, seasonings such as a tomato sauce, a bouilon and a soy sauce are also included in the food of the invention.

Further, the food composition of the invention may be a composition comprising melatonin and any additives for incorporating melatonin in any food, for example, a tablet comprising melatonin and a disintegrator; a mixture comprising melatonin and an extender such as a proteolyzate, a starch, casein and glucose; melatonin dissolved in a solvent such as an edible fat and oil, ethanol and water; and a W/O or O/W emulsifiable product comprising melatonin. Such a composition may be in the form of a powder, a tablet, an extrudate and the like.

The addition of melatonin into a food may be conducted at any suitable time. For example, melatonin may be added in a raw foodstuff before cooking such as a minced meat, a cooked food such as a bouillabaisse and a stew, or milk before preparation of yogurt. Alternatively, melatonin may be added in a food before cold storing or freezing.

An amount of melatonin to be added in a food is such that the above mentioned daily dose of melatonin is satisfied.

While the composition of the invention has been described, it will be apparent to those skilled in the art that many changes and modifications can be made thereto without departing from the spirit of the invention as set forth herein.

The invention will be describe with reference to the following examples which are included herein for the purposes of illustration and are not intended to be limiting of the invention.

### EXAMPLE 1

(1) Brain ischemia preferentially injures astroglia.

Eight to ten-week-old Wister male rats having the body weight of 250 to 300 g were used.

Under halothane anesthesia, a 24G plug was inserted from a right external carotid artery of each rat so as to arrive to an origin of its middle cerebral artery of Willis's circle through its internal carotid artery. Immediately the anesthesia was stopped and the rat was dehypnotized. After one hour, the plug was taken out under halothane anesthesia, thereby a blood flow was recanalized.

After 11 days from an onset of brain ischemia, the brain of each rat was fixed with an aqueous 4% paraformaldehyde solution to prepare a brain specimen. Then, the brain specimen was stained according to the standard ABC method using an anti-GFAP antibody (GFAP staining; astroglia were stained).

As shown in photographs A and B corresponding to an amplification of an area enclosed with a square in photograph A, astroglia in an ischemic core area was fallen out and a gliosis, an overgrowing of astroglia, was observed in its penumbra area (a lateral area of corpus striatum and a mantle of cerebral cortex). Photograph C corresponding to an amplification of an area enclosed with a square in photograph B showed that in an ischemic core area of cerebral cortex, astroglia was killed, but neuron was alive. Photograph D corresponding to an amplification of an area enclosed with a square in photograph A showed that in an penumbra area of cerebral cortex, astroglia was killed, but neuron was alive. Photographs E and F showed cerebral cortex in which → represents a neuron and →→ represents both a neuron and an astroglia. Photograph E showed that astroglia were killed, but neurons was alive. Photograph F showed that both neurons and astroglia was alive.

These results demonstrate that brain ischemia preferentially injuries astroglia before neurons, i.e. that neuron is not injured directly by brain ischemia. Accordingly, these results strongly suggest that brain edema due to brain ischemia is caused by the modification in cerebrovascular permeability resulting from the injury of astroglia and that a secondary modification in cerebrovascular permeability resulting from a secondary injury of astroglia which is caused by a secondary lowering of a cerebral blood flow caused by the original edema is involved in a “forest fire-like” extension of brain edema.

(2) Melatonin protects astroglia from the injury due do brain ischemia (in vivo).

Ischemic rats in Example 1 were divided into two groups. In the first group (non-melatonin administration group), a physiological saline solution was injected to a stomach of each rat using a sound for 10 days from the first day (24 hours after the recanalization of the blood flow). In the second group (melatonin administration group), a melatonin-containing physiological saline solution was similarly administered in an amount of 6 mg of melatonin per kg of rat.

Specimens of brain in the non-melatonin administration group (photographs A, B and C) and in the melatonin administration group (photograph D, E and F) were made in the same manner as described in Example 1. These brain specimens were stained using a GFAP staining (photographs A, B, D and E) or a MAP-2 staining with an anti-MAP-2 antibody staining neurons (photographs C and F).

Results of the GFAP staining showed that the falling out of astroglia in an ischemic core area and the gliosis in its penumbra area were observed in the non-melatonin administration group (photographs A and B corresponding to an amplification of an area enclosed with a square in photograph A), and the falling out of astroglia and the gliosis were reduced by the administration of melatonin in the melatonin administration group (photographs D and E corresponding to an amplification of an area enclosed with a square in photograph D).

Results of the MAP-2 staining showed that neurons were alive in both the non-melatonin administration group (photograph C) and the melatonin administration group (photograph F). The number of the alive neurons in the melatonin administration group was higher than that in the non-melatonin administration group.

(3) Melatonin protects a blood-brain barrier from the injury due to brain ischemia.

An IgG diapedesis area (area stained with an anti-IgG antibody) from a blood vessel in each of cerebral cortex and corpus striatum was observed using the NIH imaging system in the non-melatonin administration group and the melatonin administration group of Example 2. Results are shown in Table 1.

TABLE 1

IgG diapedesis area
(mm²; average ± SE)

| group | population | cerebral cortex | corpus striatum |
|---|---|---|---|
| non-melatonin | 11 | 14.5 ± 2.0 | 10.6 ± 3.0 |
| administration | | | |
| melatonin | 11 | 5.5 ± 4.5 | 7.2 ± 4.0 |
| administration | | | |

Table 1 demonstrates that in the non-melatonin administration group, the diapedesis of IgG into cerebral cortex and corpus striatum occurs due to brain ischemia and brain ischemia breaks a blood-brain barrier. And, it demonstrates that such a breakage of a blood-brain barrier due to brain ischemia is inhibited by the administration of melatonin in the melatonin administration group.

Results of experiment (3) together with those of experiment (2) demonstrate that brain ischemia preferentially injures astroglia leading to the breakage of a blood-brain barier and that the injury of astroglia, i.e. the breakage of a blood-brain barrier, is inhibited by melatonin.

(4) Melatonin protects astroglia from the injury caused by a drug (in vitro).

Isolation and incubation of astrocytes were conducted according to the method as described in Neuroscience, 87: 497-807, 1998. Thus, cerebral cortex of a fetal rat was removed, from which astrocytes were separated with trypsin treatment at 37° C. for 20 minutes and incubated on a 24 well culture dish containing DMEM media+10% FBS under 5% CO₂/air atmosphere at 37° C.

The injury of incubated astrocytes was induced by replacing the medium with a serum-free DMEM medium (serum-free group) or adding 6 mM of a metabolic inhibitor, 3-nitropropionic acid (3-NPA), to the medium (3-NPA group). The serum-free group and the 3-NPA group were divided into two sub-groups, respectively. To one sub-group of each group, 10 μg/ml of melatonin was added to the medium.

The number of the alive astrocytes was counted according to the neutron red assay after 3 days from the incubation in the 3-NPA group or according to the MTT (3-(4,5-dimethyl-thiozolyl)-2,5-diphenyl-tetrazolium bromide) assay after 10 days from the replacement of the medium. The test of significance was conducted by ANOVA. Results are shown in Table 2.

TABLE 2

| | number of astrocytes |
|---|---|
| | (% of control; |
| group | average ± SE) |
| non-treatment | 100 ± 5 |
| 3-NPA | 50 ± 5 |
| 3-NPA + melatonin | 63 ± 4* |
| serum-free | 34 ± 3 |
| serum-free + melatonin | 48 ± 4** |

*p < 0.05 (v.s. control)
**p < 0.01 (v.s. control)

As clear from the results in Table 2, melatonin protected astrocytes from the injury caused by 3-NPA or the injury caused by the replacement of a serum-free medium.

In conclusion, the experiments of Example 1 demonstrate that brain ischemia preferentially injures astroglia, destroys blood-brain barrier, and deteriorate vascular permeability, which deterioration causes brain edema. The experiments further demonstrate that the ischemic or poisonous injury of astroglia can be prevented and cured by melatonin, strongly suggesting that melatonin will treat and prevent brain edema caused by the first injury of astroglia or the second injury of astroglia caused by edema.

### EXAMPLE 2

Treatment/prevention of brain edema by melatonin

A plug is inserted from an internal carotid artery of a 8 to 10-week-old SD male rat so as to arrive to a branch of its middle cerebral artery, thereby the middle cerebral artery is occluded for 60 minutes.

Astroglias in a penumbra area of brain capillary is injured by brain ischemia and astroglias in other areas and neutron are injured by an ischemic metabolic disorder so that cytotoxic edema is mainly caused. Thereafter, the blood flow is recanalized by removing the plug, thereby a blood flows into the ischemia area at a stroke and a moisture is passed from an injured blood-brain barrier to a paremchyma of brain so that vasogenic edema is mainly caused.

Immediately, 6 hours and 12 hours after the recanalization, 0.1 to 1 mg/kg of melatonin is intravenously administered. Extent of brain edema is observed after 24 hours from the recanalization.

The extent of brain edema can be observed by determining the change in intracerebral moisture content using MRI according to two analysis methods; the T2 highlighted image mainly catching vasogenic edema and the scattering highlighted image mainly catching cytotoxic edema. And, it can be determined by observing a brain specimen under a microscope.

In addition, a motality and a cerebral infarcted area after orally administrating melatonin in an amount of 6 mg/kg/day for one week are determined.

1. A method for the treatment or prevention of brain edema comprising administering to a subject in need of said treatment or prevention a composition for said treatment or prevention comprising melatonin in an effective amount for said treatment or prevention.

2. A method as claimed in claim 1 wherein the composition is a pharmaceutical composition.

3. A method as claimed in claim 2 wherein melatonin is encapsulated in an encapsulating matrix or a liposome.

4. A method as claimed in claim 2 wherein the subject is suffering from brain edema.

5. A method as claimed in claim 2 wherein the subject has the risk of suffering from brain ischemia.

6. A method as claimed in claim 5 wherein the subject having the risk of suffering from brain ischemia is a subject suffering from cerebral thrombosis, cerebral embolism, cerebral infarction, cerebral hemorrhage, subarachnoid hemorrhage, transient brain ischemia, hyperlipemia, hypertension, cardiac arrest or brain contusion.

7. A method as claimed in claim 1 wherein the composition is orally administered.

8. A method as claimed in claim 1 wherein the composition is a food composition.

9. A method as claimed in claim 8 wherein the subject is suffering from brain edema.

10. A method as claimed in claim 8 wherein the subject has the risk of suffering from brain ischemia.

11. A method as claimed in claim 10 wherein the subject having the risk of suffering from brain ischemia is a subject suffering from cerebral thrombosis, cerebral embolism, cerebral infarction, cerebral hemorrhage, subarachnoid hemorrhage, transient brain ischemia, hyperlipemia, hypertension, cardiac arrest or brain contusion.

12. A method as claimed in claim 8 wherein the food composition is selected from the group consisting of food, a food stuff and a composition comprising melatonin and an additive for incorporating melatonin in food.

13. A pharmaceutical or food composition for the treatment or prevention of brain edema comprising melatonin in an effective amount for said treatment or prevention.

14. Use of melatonin in the preparation of a pharmaceutical or food composition for the treatment or prevention of brain edema comprising melatonin in an effective amount for said treatment or prevention.
